(19) United States
(12) Patent Application Publication
(10) Pub. No.: US 2013/0072799 A1
(43) Pub. Date: Mar. 21, 2013

(54) ULTRASOUND DIAGNOSTIC APPARATUS AND ULTRASOUND IMAGE GENERATING METHOD

(71) Applicant: Tomoo SATO, Kanagawa (JP)

(72) Inventor: Tomoo SATO, Kanagawa (JP)

(21) Appl. No.: 13/599,595

(22) Filed: Aug. 30, 2012

(30) Foreign Application Priority Data
Sep. 16, 2011 (JP) 2011-203231

(51) Int. Cl. A61B 8/14

(52) U.S. Cl. 600/447

(57) ABSTRACT

An ultrasound diagnostic apparatus includes an array transducer, a transmission driver for transmitting an ultrasonic beam from the array transducer toward a subject, a reception circuit for processing a pair of reception signals in a pair of available channels of the array transducer having received an ultrasonic echo produced by the subject, with the pair of available channels being those channels which belong to channels simultaneously made available for reception of the ultrasonic echo and are arranged symmetrically about a transmit beam axis, so as to generate reception data in one piece, and outputting the generated reception data as reception data in each of the pair of available channels, and an image generating unit for generating an ultrasound image based on the reception data outputted from the reception circuit.

### BACKGROUND OF THE INVENTION

The present invention relates to an ultrasound diagnostic apparatus and an ultrasound image generating method, and particularly to an ultrasound diagnostic apparatus adapted to generate an ultrasound image based on the reception data which is acquired by amplification and analog/digital conversion performed in a reception signal processing unit on a reception signal outputted from an array transducer having received an ultrasonic echo produced by a subject.

In the medical field, ultrasound diagnostic apparatus employing ultrasound images have already been put to practical use. A typical ultrasound diagnostic apparatus for medical use transmits an ultrasonic beam from an array transducer of an ultrasound probe toward the inside of a subject, receives an ultrasonic echo from the subject on the array transducer, and electrically processes a reception signal corresponding to the received echo in an apparatus body so as to generate an ultrasound image.

As an example, JP 4-232888 A discloses an ultrasonic diagnostic system performing reception focusing, on which reception signals outputted from an array transducer having received ultrasonic echo components are each amplified by a preamplifier and subjected to analog/digital conversion by an analog/digital converter to obtain digital reception data, and the obtained digital data are matched to one another in phase by imparting adequate delays to them, and as such added to one another so as to carry out the reception focusing.

According to the reception focusing process as disclosed, a sound ray signal is obtained with a well-focused ultrasonic echo, and the B-mode image signal which is tomographic image information on the inside of a subject is generated based on a plurality of sound ray signals obtained in a region under diagnosis.

In such ultrasonography as conducted on the above system, an ultrasonic beam is attenuated while traveling within the subject, so that the ultrasonic beam which reaches a deeper site in the subject will have a lower intensity. In addition, an ultrasonic echo reflected by any site in the subject toward the ultrasound probe is attenuated while traveling within the subject. Moreover, the center frequency is shifted toward the low frequency side due to the frequency-dependent attenuation. As a consequence, a reception signal outputted from the array transducer will vary in amplitude with measurement depth.

The analog/digital converter is thus desired to have so broad a dynamic range as to allow reception signals throughout a measurement region, ranging from a reception signal with a larger amplitude corresponding to a shallower zone in the measurement region to a reception signal with a smaller amplitude corresponding to a deeper zone, to be analog/digital converted with a good resolution. In the conventional ultrasound diagnostic apparatus, however, an analog/digital converter used is inadequate in dynamic range.

In order to cope with such a problem, JP 2-004346 A, for instance, discloses the ultrasound diagnostic apparatus in which a pair of amplifiers with different gains are connected in parallel with an ultrasound transducer, and the reception signals as amplified by the amplifiers are individually subjected to analog/digital conversion by analog/digital converters, and then combined together. The disclosed configuration makes it possible to subject reception signals with a wide variety of amplitudes to favorable analog/digital conversion so as to generate an ultrasound image of high quality.

The above configuration, however, requires that a pair of amplifiers and a pair of analog/digital converters should be provided for each of multiple channels constituted of an array transducer, which will increase the number of components and power consumption, and complicate the structure of an apparatus.

### SUMMARY OF THE INVENTION

The present invention, as being made in order to dissolve such problems as above with the prior art, has an object of providing an ultrasound diagnostic apparatus and an ultrasound image generating method allowing the generation of an ultrasound image of high quality with a simple configuration and low power consumption.

An ultrasound diagnostic apparatus according to the present invention comprises: an array transducer; a transmission driver for transmitting an ultrasonic beam from the array transducer toward a subject; a reception circuit for processing a pair of reception signals in a pair of available channels of the array transducer having received an ultrasonic echo produced by the subject, with the pair of available channels being those channels which belong to channels simultaneously made available for reception of the ultrasonic echo and are arranged symmetrically about a transmit beam axis, so as to generate reception data in one piece, and outputting the generated reception data as reception data in each of the pair of available channels; and an image generating unit for generating an ultrasound image based on the reception data outputted from the reception circuit.

An ultrasound image generating method according to the present invention comprises the steps of: transmitting an ultrasonic beam from an array transducer toward a subject; processing a pair of reception signals in a pair of available channels of the array transducer having received an ultrasonic echo produced by the subject, with the pair of available channels being those channels which belong to channels simultaneously made available upon reception of the ultrasonic echo and are arranged symmetrically about a transmit beam axis, so as to generate reception data in one piece and use the generated reception data as reception data in each of the pair of available channels; and generating an ultrasound image based on the reception data.

### BRIEF DESCRIPTION OF THE DRAWINGS

FIG. 1 is a block diagram showing a configuration of an ultrasound diagnostic apparatus according to Embodiment 1 of the present invention;

FIGS. 2A and 2B show an internal structure of a reception circuit as used in Embodiment 1 in measurement for a measurement depth zone at a shallow location and in measurement for a measurement depth zone at a deep location, respectively;

FIG. 3 is a diagram schematically showing delay times of an ultrasonic echo reaching from a reflection source on a transmit beam axis to ultrasound transducers of individual channels;

FIG. 4 is a block diagram showing an internal structure of a reception circuit as used in Embodiment 2;

FIG. 5 is a block diagram showing an internal structure of a reception circuit as used in Embodiment 3;

FIG. 6 is a diagram schematically showing the analog/digital conversion ranges of a pair of analog/digital converters used in Embodiment 3;

FIG. 7 is a diagram illustrating the state of available channels in a reference case; and

FIG. 8 is a diagram illustrating the state of available channels in Embodiment 3.

### DETAILED DESCRIPTION OF THE INVENTION

In the following, embodiments of the present invention are described in reference to the accompanying drawings.

### Embodiment 1

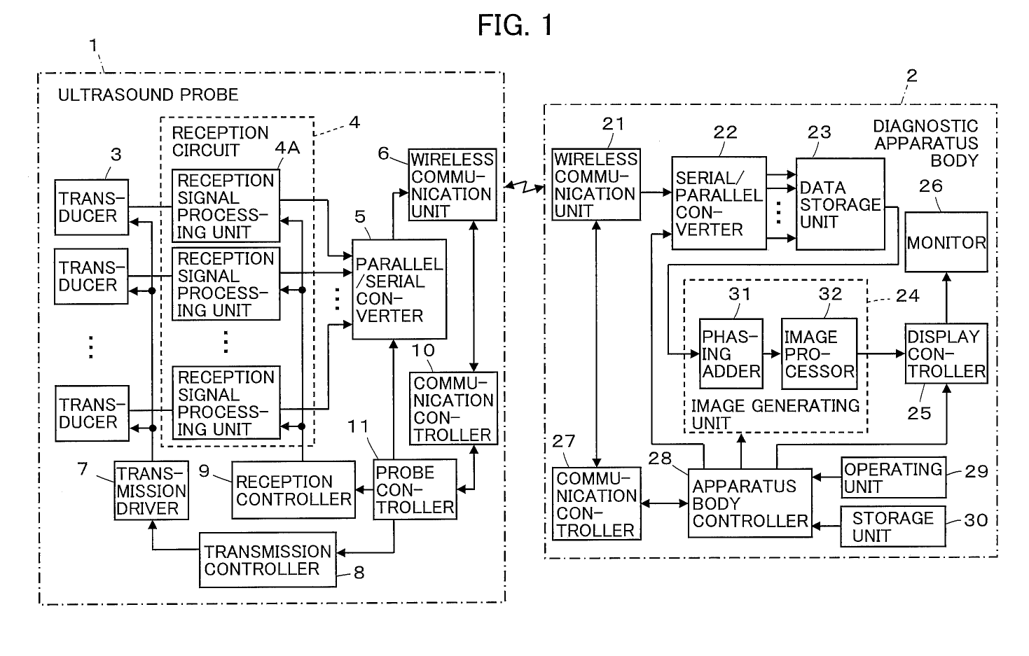

FIG. 1 shows a configuration of an ultrasound diagnostic apparatus according to Embodiment 1. The ultrasound diagnostic apparatus as shown includes an ultrasound probe 1 and a diagnostic apparatus body 2 connected with the ultrasound probe 1 through wireless communication.

The ultrasound probe 1 has a plurality of ultrasound transducers 3 one- or two-dimensionally arrayed, and the ultrasound transducers 3 are connected to a reception circuit 4. The reception circuit 4 is connected to a wireless communication unit 6 via a parallel/serial converter 5. The reception circuit 4 comprises a plurality of reception signal processing units 4A connected with the ultrasound transducers 3, respectively. The ultrasound transducers 3 are connected with a transmission controller 8 via a transmission driver 7, while the reception signal processing units 4A in the reception circuit 4 are connected with a reception controller 9. The wireless communication unit 6 is connected with a communication controller 10. The parallel/serial converter 5, the transmission controller 8, the reception controller 9, and the communication controller 10 are connected with a probe controller 11.

Each of the ultrasound transducers 3 transmits ultrasound in accordance with a driving signal fed from the transmission driver 7, and receives an ultrasonic echo from a subject so as to output a reception signal. Each ultrasound transducer 3 is comprised of a vibrating element having a piezoelectric body and electrodes formed at both ends of the piezoelectric body, with examples of the material for the body including a piezoelectric ceramic typified by lead zirconate titanate (PZT), a polymeric piezoelectric material typified by polyvinylidene fluoride (PVDF), and a piezoelectric single crystal typified by lead magnesium niobate-lead titanate solid solution (PMN-PT).

If a pulsed voltage or a continuous wave voltage is applied across the electrodes of the vibrating element as above, the piezoelectric body expands and contracts, and ultrasound in pulse or continuous wave form is generated from the vibrating element. Ultrasound waves generated from the individual vibrating elements are synthesized into an ultrasonic beam. In addition, each vibrating element expands and contracts during the reception of propagating ultrasound to generate an electric signal, and the electric signal is outputted as a reception signal representing the reception of ultrasound.

The transmission driver 7 includes, for instance, a plurality of pulse generators, and is adapted to modify, based on the transmission delay pattern as selected by the transmission controller 8, the delay amounts of the driving signals to be fed to the ultrasound transducers 3 so that ultrasounds transmitted from the ultrasound transducers 3 may form so wide an ultrasonic beam as to cover a specified tissue area in a subject, and then feed the driving signals to the transducers 3.

Each reception signal processing unit 4A in the reception circuit 4 generates a complex baseband signal by subjecting a reception signal outputted from the corresponding ultrasound transducer 3 to quadrature detection or quadrature sampling under the control of the reception controller 9, then performs sampling of the complex baseband signal to generate sample data including information on the tissue area, and feeds the sample data to the parallel/serial converter 5. The reception signal processing units 4A may generate sample data by subjecting the data as acquired by the sampling of the complex baseband signal to data compression for low bit rate coding.

The parallel/serial converter 5 converts the sample data in parallel form as generated by the reception signal processing units 4A into serial sample data.

The wireless communication unit 6 transmits the serial sample data by modulating a carrier based on the serial sample data to generate a transmission signal, and feeding the transmission signal to an antenna to thereby transmit radio waves from the antenna. Usable modulation methods include amplitude shift keying (ASK), phase shift keying (PSK), quadrature phase shift keying (QPSK), and sixteen quadrature amplitude modulation (16QAM).

The wireless communication unit 6 communicates with the diagnostic apparatus body 2 in a wireless manner so as not only to transmit sample data to the diagnostic apparatus body 2, but receive various control signals from the diagnostic apparatus body 2 to output the received control signals to the communication controller 10. The communication controller 10 controls the wireless communication unit 6 so that transmission of sample data may be performed at the radio field intensity for transmission as specified by the probe controller 11, and outputs the control signals as received by the wireless communication unit 6 to the probe controller 11.

The probe controller 11 controls individual components of the ultrasound probe 1 based on various control signals transmitted from the diagnostic apparatus body 2.

The ultrasound probe 1 has a battery included therein (not shown), from which power is fed to individual circuits in the ultrasound probe 1.

The ultrasound probe 1 may be an external probe, such as of a linear scanning type, of a convex scanning type, and of a sector scanning type, or a probe for endoscopic ultrasonography, such as of a radial scanning type.

The diagnostic apparatus body 2 has a wireless communication unit 21 connected to a data storage unit 23 via a serial/parallel converter 22, with the data storage unit 23 being connected to an image generating unit 24. The image generating unit 24 is connected to a monitor 26 via a display controller 25. The wireless communication unit 21 is also connected with a communication controller 27, and the serial/parallel converter 22, the data storage unit 23, the image generating unit 24, the display controller 25 and the communication controller 27 are connected with an apparatus body controller 28. The apparatus body controller 28 is in turn connected with an operating unit 29 used by an operator to perform input operations, and a storage unit 30 for storing operational programs.

The wireless communication unit 21 communicates with the ultrasound probe 1 in a wireless manner so as to transmit various control signals to the ultrasound probe 1. In addition, the wireless communication unit 21 outputs serial sample data by demodulating a signal received by an antenna.

The communication controller 27 controls the wireless communication unit 21 so that transmission of various control signals may be performed at the radio field intensity for transmission as specified by the apparatus body controller 28.

The serial/parallel converter 22 converts serial sample data outputted from the wireless communication unit 21 into parallel sample data. The data storage unit 23 is comprised of a memory, a hard disk or the like, and adapted to store the sample data as converted by the serial/parallel converter 22 for at least one frame.

The image generating unit 24 subjects the sample data as read frame by frame from the data storage unit 23 to reception focusing so as to generate an image signal representing an ultrasound diagnostic image. The image generating unit 24 includes a phasing adder 31 and an image processor 32.

The phasing adder 31 selects, in accordance with the reception direction as specified in the apparatus body controller 28, one reception delay pattern from among those stored in advance, and provides complex baseband signals represented by the sample data with their respective delays based on the selected reception delay pattern, then adds the delayed signals to thereby perform the reception focusing. The reception focusing allows a baseband signal (sound ray signal) as a well-focused ultrasonic echo.

The image processor 32 generates the B-mode image signal which is tomographic image information on a tissue in the subject based on a sound ray signal generated by the phasing adder 31. The image processor 32 includes a sensitivity time control (STC) device and a digital scan converter (DSC). The STC device corrects the sound ray signal for attenuation due to distance in accordance with the depth of the position where ultrasound was reflected. The DSC subjects the sound ray signal as corrected by the STC device to the conversion (raster conversion) into an image signal compatible with the conventional television signal scanning method, and to such image processing as grayscaling as required, so as to generate a B-mode image signal.

The display controller 25 controls the monitor 26 based on an image signal generated by the image generating unit 24 to display an ultrasound diagnostic image. The monitor 26 includes a display device such as an LCD, and is adapted to display an ultrasound diagnostic image under the control of the display controller 25.

The apparatus body controller 28 controls individual components of the diagnostic apparatus body 2 based on various instruction signals and the like inputted by an operator from the operating unit 29.

In the diagnostic apparatus body 2 as configured above, the serial/parallel converter 22, the image generating unit 24, the display controller 25, the communication controller 27, and the apparatus body controller 28 are implemented by a CPU associated with operational programs for giving the CPU instructions on various kinds of processing, while the above components may also be implemented by a digital circuitry. The operational programs are stored in the storage unit 30. The storage unit 30 may include as a recording medium a flexible disk, MO, MT, RAM, CD-ROM, DVD-ROM, an SD card, a CF card, a USB memory or the like besides a built-in hard disk. A server is also usable for storing operational programs.

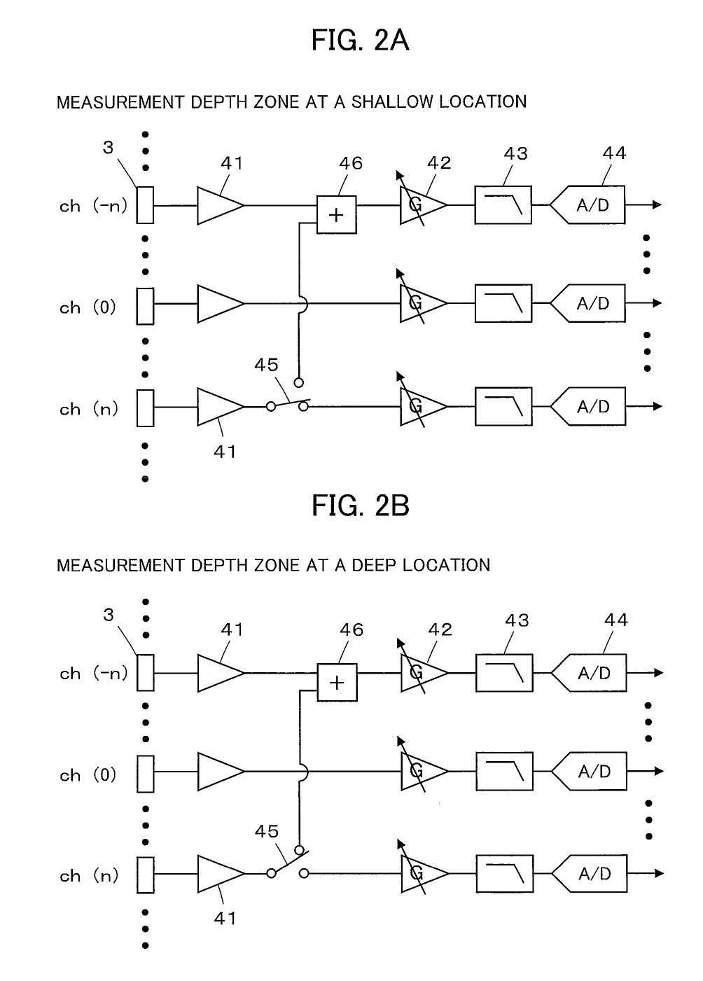

FIG. 2A shows an internal structure of the reception circuit 4 in the ultrasound probe 1. The reception circuit 4 comprises the reception signal processing units 4A corresponding to the ultrasound transducers 3, with each reception signal processing unit 4A being so constructed that a preamplifier 41 and a variable gain amplifier 42 for amplifying a reception signal outputted from the corresponding ultrasound transducer 3, a low pass filter 43 for removing high frequency components not used for signal detection from the reception signal, and an analog/digital converter 44 for analog/digital conversion of the reception signal are connected in series in this order.

The ultrasound transducers 3 constitute multiple channels, and a specified number of channels symmetric about the transmit beam axis are simultaneously made available during the reception of an ultrasonic echo. The reception circuit 4 is adapted to add together the reception signals as obtained in a pair of channels, respectively, with the pair of channels being arranged symmetrically about the transmit beam axis. As an example, assuming that a channel ch(0) in FIG. 2A coincides with a transmit beam axis, a selector switch 45 is connected on the output side of the preamplifier 41 in a channel ch(n), and an adder 46 is connected on the output side of the preamplifier 41 in a channel ch(−n) arranged symmetrically to the channel ch(n) about the channel ch(0). It can be determined according to the action of the selector switch 45 whether or not to add together the reception signals as obtained at the ultrasound transducers 3 in the channels ch(n) and ch(−n) and amplified by the corresponding preamplifiers 41, respectively.

For a zone at a measurement depth for ultrasonography smaller than a specified measurement depth D0, the selector switch 45 is set as shown in FIG. 2A so that the channels ch(n) and ch(−n) may not be connected with each other. On the other hand, the selector switch 45 is set as shown in FIG. 2B so that the channels ch(n) and ch(−n) may be connected with each other for a zone at a measurement depth equal to or larger than the specified measurement depth D0.

In the case of a measurement depth zone at a shallower location, reception signals obtained at the ultrasound transducers 3 each have a higher intensity, so that analog/digital conversion with a high resolution is possible to perform on the reception signals in the individual channels even if the signals as such are processed in the corresponding reception signal processing units 4A independently of each other. In the case of a measurement depth zone at a deeper location, reception signals obtained at the ultrasound transducers 3 each have a lower intensity, so that analog/digital conversion is performed on a signal produced by adding together the reception signals in the channels ch(n) and ch(−n) which are symmetric about the transmit beam axis.

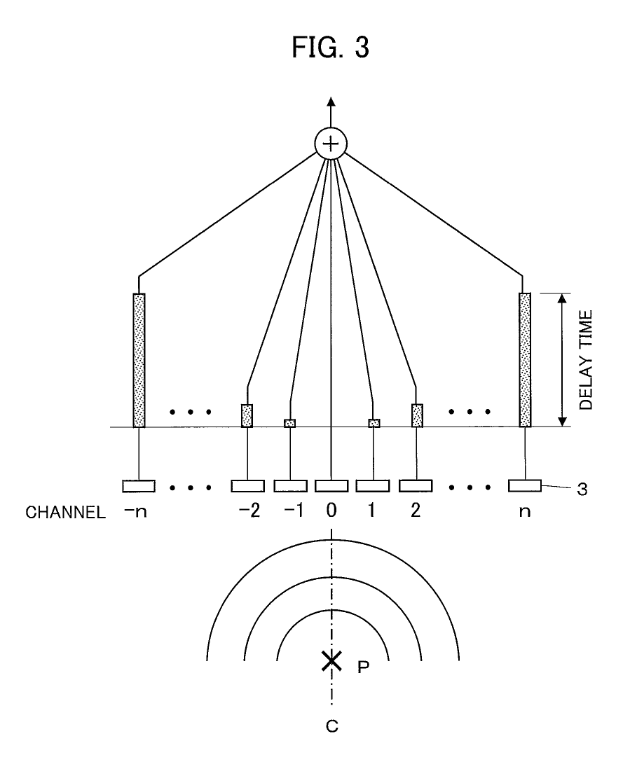

As shown in FIG. 3, similar to the intensity of an ultrasonic echo from a reflection source P on a transmit beam axis C that varies almost in an axially symmetric manner about the transmit beam axis C, the time in which the ultrasonic echo reaches the ultrasound transducers 3 in the individual channels varies in an axially symmetric manner about the transmit beam axis C. Consequently, a reception signal with a twice-higher intensity can be produced solely by adding together the reception signals in a pair of channels arranged symmetrically about the transmit beam axis C that are synchronously taken, and the reception signal thus produced can be inputted to the analog/digital converter 44 to subject it to analog/digital conversion with a high resolution.

In the above, the character “n” represents any natural number.

Operations of the apparatus of Embodiment 1 are detailed below.

The specified measurement depth D0 is stored in advance in the probe controller 11 of the ultrasound probe 1.

The ultrasound transducers 3 initially transmit ultrasound in accordance with driving signals fed from the transmission driver 7 of the ultrasound probe 1, then the reception signals as outputted from the ultrasound transducers 3 having received an ultrasonic echo from a subject are fed to the corresponding reception signal processing units 4A of the reception circuit 4, respectively.

Under the control of the probe controller 11, the reception controller 9 sets the selector switch 45 as shown in FIG. 2A for a zone at a measurement depth smaller than the specified measurement depth D0, that is to say, until time corresponding to the specified measurement depth D0 elapses from the transmission of ultrasound so that the channels ch(n) and ch(−n) which are symmetric about the transmit beam axis may not be connected with each other. As a result, reception signals in the individual channels as simultaneously made available during reception undergo analog/digital conversion in the corresponding reception signal processing units 4A independently of each other, and are outputted as sample data.

For a zone at a measurement depth equal to or larger than the specified measurement depth D0, that is to say, after the time corresponding to the specified measurement depth D0 has elapsed since the transmission of ultrasound, the reception controller 9 sets the selector switch 45 as shown in FIG. 2B so that the channels ch(n) and ch(−n) which are symmetric about the transmit beam axis may be connected with each other. The reception signals in the channels ch(n) and ch(−n) are then added together to produce a reception signal with a twice-higher intensity. The reception signal thus produced undergoes analog/digital conversion at the analog/digital converter 44 in the channel ch(−n), for instance, and is outputted as sample data both in the channel ch(n) and in the channel ch(−n).

The operation as described above is performed for each pair of available channels simultaneously made available during reception that are arranged symmetrically about the transmit beam axis, so that reception signals in each pair of available channels are added together before analog/digital conversion, with the reception signal having undergone analog/digital conversion being outputted as sample data in the relevant pair of available channels.

The thus generated sample data is made serial at the parallel/serial converter 5 before being transmitted from the wireless communication unit 6 to the diagnostic apparatus body 2 in a wireless manner. The sample data as received at the wireless communication unit 21 of the diagnostic apparatus body 2 is converted at the serial/parallel converter 22 into parallel data, then stored in the data storage unit 23. The stored sample data is read from the data storage unit 23 frame by frame, an image signal is generated by the image generating unit 24, and an ultrasound diagnostic image is displayed on the monitor 26 by the display controller 25 based on the image signal.

As described above, for a measurement depth zone at a deep location, reception data in one piece is generated by performing analog/digital conversion on the reception signal as produced by adding together the reception signals in the channels ch(n) and ch(−n) which are symmetric about the transmit beam axis, and the generated reception data is outputted as reception data both in the channel ch(n) and in the channel ch(−n). Consequently, analog/digital conversion of a reception signal with a high resolution is possible even for a measurement depth zone at a deep location producing an ultrasonic echo with a low intensity. Moreover, only one preamplifier 41 and one analog/digital converter 44 need to be provided for each channel, which allows the generation of an ultrasound image of high quality with a simple configuration and low power consumption.

### Embodiment 2

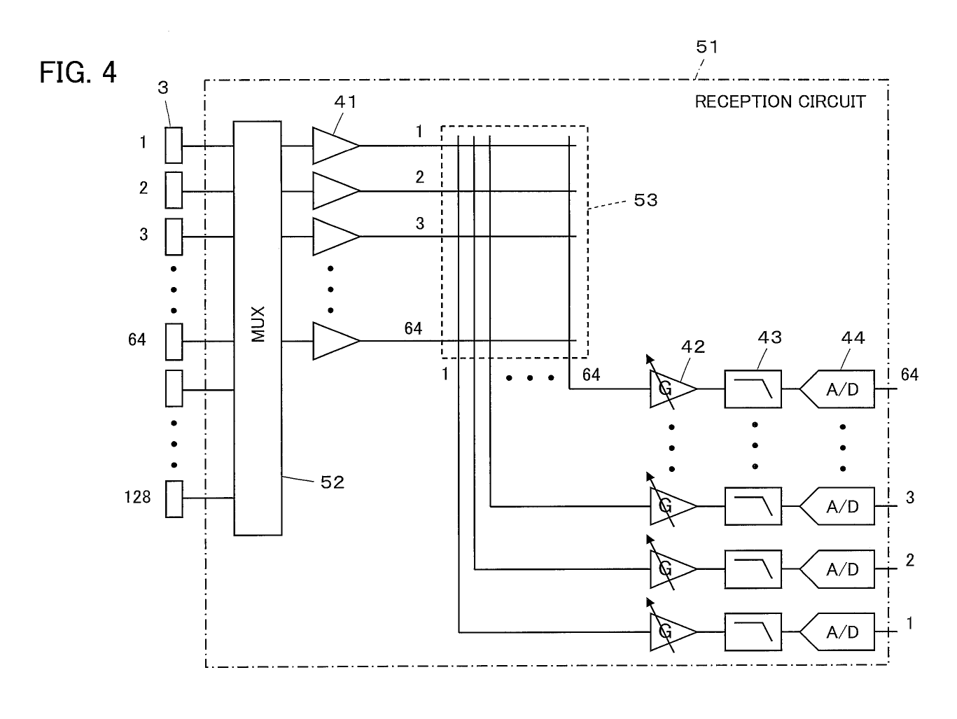

FIG. 4 shows a configuration of a reception circuit 51 in an ultrasound diagnostic apparatus according to Embodiment 2. The reception circuit 51 includes a multiplexer 52 connected with an array transducer constituting 128 channels, for instance, and also connected to preamplifiers 41 which are 64 in number. The 64 preamplifiers 41 are connected to 64 variable gain amplifiers 42 through a crosspoint switch 53, while low pass filters 43 and analog/digital converters 44 are sequentially connected with the variable gain amplifiers 42.

The multiplexer 52 is able to make a maximum of 64 channels simultaneously available by selectively connecting 64 out of 128 ultrasound transducers 3 to the 64 preamplifiers 41.

The crosspoint switch 53 is adapted to connect each of the 64 preamplifiers 41 to one of the 64 variable gain amplifiers 42 in a selective manner.

The ultrasound diagnostic apparatus according to Embodiment 2 is identical in configuration to the ultrasound diagnostic apparatus of Embodiment 1 as shown in FIG. 1 except for the reception circuit 51.

In ultrasonography, it is normal to transmit and receive ultrasound while scanning a specified number of simultaneous available channels, which causes the transmit beam axis to change in position at every scanning operation. In Embodiment 2, the crosspoint switch 53 controls simultaneous available channels every time the multiplexer 52 selects them so that a pair of simultaneous available channels symmetric about the transmit beam axis may or may not be connected with each other.

To be more specific, in the case of a zone at a measurement depth smaller than the specified measurement depth D0, the crosspoint switch 53 functions so that a pair of channels symmetric about the transmit beam axis may not be connected with each other and the reception signals in the channels may accordingly undergo analog/digital conversion independently of each other. In the case of a zone at a measurement depth equal to or larger than the specified measurement depth D0, the crosspoint switch 53 functions so that a pair of channels symmetric about the transmit beam axis may be connected with each other so as to perform analog/digital conversion on the reception signals in the channels after adding together the signals.

Even though ultrasound transmission and reception are performed while scanning simultaneous available channels, it is thus consistently possible that, for a measurement depth zone at a deep location, analog/digital conversion is performed on the reception signal as produced by adding together the reception signals in a pair of channels symmetric about the transmit beam axis so as to generate reception data in one piece, and the generated reception data is outputted as reception data in each of the channels. Consequently, an ultrasound image of high quality is allowed to be generated.

While the reception circuit 51 in Embodiment 2 is a 64-channel circuit and is connected with an array transducer constituting 128 channels, the mentioned numbers of channels, and bit number as well, are merely examples not limiting the present invention.

### Embodiment 3

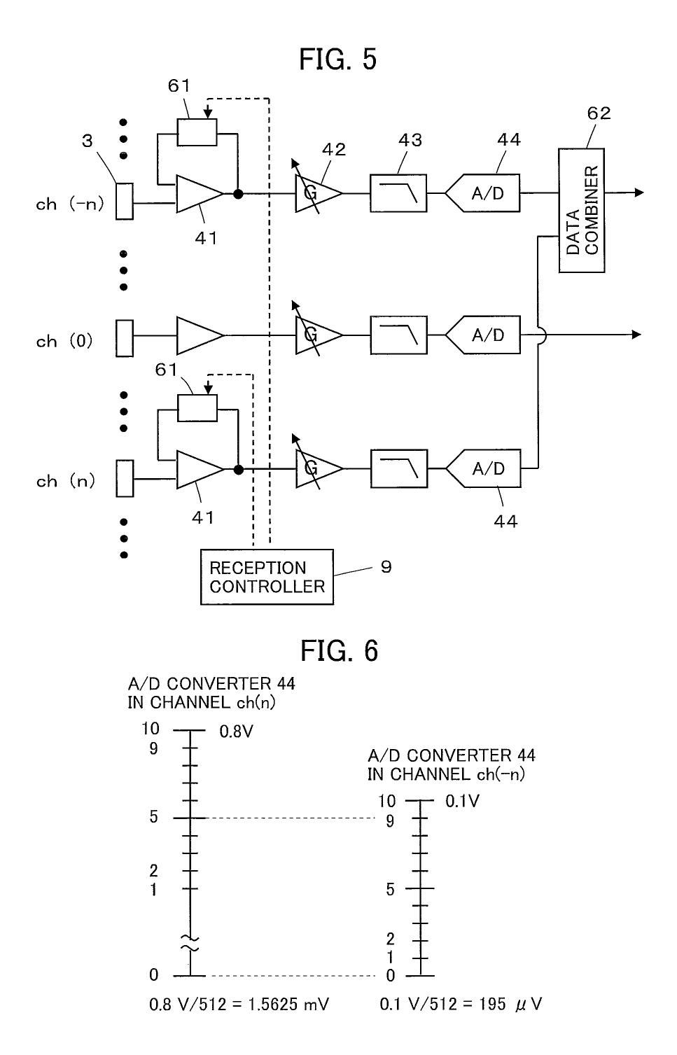

FIG. 5 shows a configuration of a reception circuit in an ultrasound diagnostic apparatus according to Embodiment 3.

With each of ultrasound transducers 3, a preamplifier 41, a variable gain amplifier 42, a low pass filter 43, and an analog/digital converter 44 are connected in series in this order.

The ultrasound transducers 3 constitute multiple channels, and a specified number of channels symmetric about the transmit beam axis are simultaneously made available during the reception of an ultrasonic echo. The reception circuit as shown is adapted to allow analog/digital conversion with different dynamic ranges in a pair of available channels arranged symmetrically about the transmit beam axis.

As an example, assuming that a channel ch(0) in FIG. 5 coincides with a transmit beam axis, a gain changing circuit 61 is connected in parallel with the preamplifier 41 in each of channels ch(n) and ch(−n) arranged symmetrically about the channel ch(0), with the gain changing circuits 61 being also connected with the reception controller 9. In addition, a data combiner 62 is connected on the output side of the analog/digital converters 44 in the channels ch(n) and ch(−n).

In the above, the character “n” represents any natural number.

The gain changing circuits 61 and the reception controller 9 constitute a gain changer, while the data combiner 62 constitutes a data combiner.

As shown in FIG. 3, similar to the intensity of an ultrasonic echo from the reflection source P on the transmit beam axis C that varies almost in an axially symmetric manner about the transmit beam axis C, the time in which the ultrasonic echo reaches the ultrasound transducers 3 in the individual channels varies in an axially symmetric manner about the transmit beam axis C. In Embodiment 3, the reception controller 9 controls the gain changing circuits 61 which are connected in parallel with the preamplifiers 41 in the channels ch(n) and ch(−n) as arranged symmetrically about the transmit beam axis C, respectively, so that the preamplifiers 41 may differ in gain from each other.

During the reception of an ultrasonic echo, the reception signals in the channels ch(n) and ch(−n) symmetric about the transmit beam axis are amplified by the preamplifiers 41 as set at different gains, then undergo analog/digital conversion at the analog/digital converters 44. A pair of data pieces resulting from the analog/digital conversion are combined together by the data combiner 62, with the combined data being outputted as reception data both in the channel ch(n) and in the channel ch(−n).

The operation as above is performed for each pair of available channels simultaneously made available during reception that are arranged symmetrically about the transmit beam axis, so that reception signals in each pair of available channels are amplified by the preamplifiers 41 as set at different gains, and undergo analog/digital conversion at the analog/digital converters 44, with the resulting pair of data pieces being combined together by the data combiner 62 so as to output the combined data as sample data in the relevant pair of available channels.

If the preamplifier 41 in the channel ch(−n) is set at a gain eight times as high as the gain of the preamplifier 41 in the channel ch(n) and the dynamic range of analog/digital conversion in the channel ch(n) is from 0 to 0.8 V, for instance, the dynamic range of analog/digital conversion in the channel ch(−n) is from 0 to 0.1 V as shown in FIG. 6. Assuming that the analog/digital converters 44 as used for the channels each have an effective bit length of 512 bits, analog/digital conversion will be performed with a resolution of 0.8 V/512=1.5625 mV in the channel ch(n), and with a resolution of 0.1 V/512=195 μV in the channel ch(−n).

It is thus possible with respect to the substantially identical reception signals as obtained in the channels ch(n) and ch(−n), respectively, that a signal component on the high output level side undergo analog/digital conversion in the channel ch(n) with a first dynamic range (0 to 0.8 V), and a signal component on the low output level side in the channel ch(−n) with a second dynamic range (0 to 0.1 V), and data pieces resulting from such analog/digital conversion are combined together by the data combiner 62.

Consequently, analog/digital conversion can be carried out with a higher resolution than the analog/digital conversion which is performed in a single channel over the entire output level of a reception signal. In other words, an ultrasound image of high quality can be attained even with the analog/digital converter whose effective bit length is short.

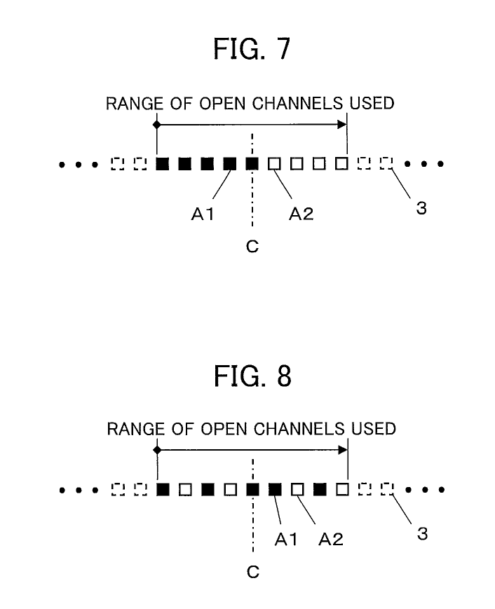

Channels A1 represented in FIGS. 7 and 8 by black squares, in which analog/digital conversion is performed with a first dynamic range, and channels A2 represented by white squares, in which analog/digital conversion is performed with a second dynamic range, may be arranged separately on both sides of the transmit beam axis C, that is to say, the channels A1 may be on one side and the channels A2 on the other as shown in FIG. 7. Taking account of a slight flaw in the axial symmetry of an ultrasonic echo about the transmit beam axis C, however, it is preferred that the channels A1 and the channels A2 are distributed on both sides of the transmit beam axis C as shown in FIG. 8. Such distribution of channels allows an ultrasound image of good reproducibility.

In Embodiments 1 through 3 as described above, the ultrasound probe 1 and the diagnostic apparatus body 2 are connected with each other through wireless communication, although the present invention is not limited thereto. It is also possible to connect the ultrasound probe 1 with the diagnostic apparatus body 2 via a connection cable. In that case, the wireless communication unit 6 and the communication controller 10 of the ultrasound probe 1, the wireless communication unit 21 and the communication controller 27 of the diagnostic apparatus body 2, and so forth are unnecessary.

What is claimed is:

1. An ultrasound diagnostic apparatus comprising:
an array transducer;
a transmission driver for transmitting an ultrasonic beam from the array transducer toward a subject;
a reception circuit for processing a pair of reception signals in a pair of available channels of the array transducer having received an ultrasonic echo produced by the subject, with the pair of available channels being those channels which belong to channels simultaneously made available for reception of the ultrasonic echo and are arranged symmetrically about a transmit beam axis, so as to generate reception data in one piece, and outputting the generated reception data as reception data in each of the pair of available channels; and
an image generating unit for generating an ultrasound image based on the reception data outputted from the reception circuit.

2. The ultrasound diagnostic apparatus according to claim 1, wherein the reception circuit generates the reception data in one piece by performing analog/digital conversion on the pair of reception signals in the pair of available channels after adding together the pair of reception signals.

3. The ultrasound diagnostic apparatus according to claim 2, wherein, for a zone at a measurement depth smaller than a specified measurement depth, the reception circuit subjects the pair of reception signals in the pair of available channels to analog/digital conversion independently of each other so as to generate reception data in the pair of available channels.

4. The ultrasound diagnostic apparatus according to claim 2, wherein the reception circuit includes two or more preamplifiers each for amplifying a reception signal outputted from the array transducer, two or more analog/digital converters each for analog/digital conversion of a reception signal, and a crosspoint switch for interconnecting the preamplifiers and the analog/digital converters in a selective manner.

5. The ultrasound diagnostic apparatus according to claim 3, wherein the reception circuit includes two or more preamplifiers each for amplifying a reception signal outputted from the array transducer, two or more analog/digital converters each for analog/digital conversion of a reception signal, and a crosspoint switch for interconnecting the preamplifiers and the analog/digital converters in a selective manner.

6. The ultrasound diagnostic apparatus according to claim 1, wherein the reception circuit subjects one reception signal out of the pair of reception signals in the pair of available channels to analog/digital conversion with a first dynamic range, and another reception signal to analog/digital conversion with a second dynamic range different from the first dynamic range, then combines the reception signals together so as to generate the reception data in one piece.

7. The ultrasound diagnostic apparatus according to claim 6, wherein the reception circuit includes two or more preamplifiers each for amplifying a reception signal in a corresponding channel, two or more analog/digital converters for analog/digital conversion of reception signals amplified by the preamplifiers, respectively, a gain changer for changing gains of two out of the preamplifiers that are corresponding to the pair of available channels so that the gains may be different from each other, and a data combiner for combining together a pair of data pieces resulting from the analog/digital conversion at two out of the analog/digital converters that are corresponding to the pair of available channels so as to generate the reception data in one piece.

8. The ultrasound diagnostic apparatus according to claim 6, wherein the reception circuit causes, out of simultaneously made available channels which are used upon reception, channels in which a reception signal is subjected to analog/digital conversion with the first dynamic range and channels in which a reception signal is subjected to analog/digital conversion with the second dynamic range to be distributed on both sides of a transmit beam axis.

9. The ultrasound diagnostic apparatus according to claim 7, wherein the reception circuit causes, out of simultaneously made available channels which are used upon reception, channels in which a reception signal is subjected to analog/digital conversion with the first dynamic range and channels in which a reception signal is subjected to analog/digital conversion with the second dynamic range to be distributed on both sides of a transmit beam axis.

10. An ultrasound image generating method comprising the steps of:
transmitting an ultrasonic beam from an array transducer toward a subject;
processing a pair of reception signals in a pair of available channels of the array transducer having received an ultrasonic echo produced by the subject, with the pair of available channels being those channels which belong to channels simultaneously made available upon reception of the ultrasonic echo and are arranged symmetrically about a transmit beam axis, so as to generate reception data in one piece and use the generated reception data as reception data in each of the pair of available channels; and
generating an ultrasound image based on the reception data.
